(19) United States
(12) Patent Application Publication
(10) Pub. No.: US 2026/0041557 A1
(43) Pub. Date: Feb. 12, 2026

(54) IMPLANT FOR REPLACING OR FILLING A DEFECT OF A FLAT BONE AND METHOD FOR PRODUCING SUCH AN IMPLANT

(71) Applicant: Karl Leibinger Asset Management GmbH & Co. KG, Mühlheim an der Donau (DE)

(72) Inventors: Adem Aksu, VS-Schwenningen (DE); Lena Schaarschmidt, Tuttlingen (DE); Frank Reinauer, Emmingen-Liptingen (DE); Tobias Wolfram, Dreieich (DE)

(21) Appl. No.: 19/293,456

(22) Filed: Aug. 7, 2025

(30) Foreign Application Priority Data
Aug. 8, 2024 (DE) 102024122716.8

(51) Int. Cl. A61F 2/28; A61L 27/04; A61L 27/10; A61L 27/26; C08L 23/06; C08L 71/00

(52) U.S. Cl. CPC A61F 2/2875; A61L 27/04; A61L 27/10; A61L 27/26; C08L 23/06; C08L 71/00; A61F 2002/2835; A61F 2002/2878

(57) ABSTRACT

An implant (X) for replacing or filling a defect of a flat bone has a flat shape with a first side (X1) and an opposite second side (X2). The implant (X) includes a first layer (S1) formed of non-porous polyetheretherketone or polyethylene, which has such a layer thickness that the shape of the implant (X) is established by the shape of the first layer (S1). The implant (X) includes, in at least some portions on the first side (X1), a second layer (S2) formed of a porous polymer which is not polyaryletherketone or polyetheretherketone. A method for producing such an implant (X) is also provided.

### CROSS-REFERENCE TO RELATED APPLICATION

The present application is related and has right of priority to German Patent Application No. DE102024122716.8 filed on Aug. 8, 2024, which is incorporated by reference in its entirety for all purposes.

### TECHNICAL FIELD

The invention relates generally to an implant for replacing or filling a defect of a flat bone, in the human or animal body. The invention further relates generally to a method for producing such an implant.

### BACKGROUND

Implants are known from the prior art, which completely replace a flat bone or are intended for filling a defect of such a bone. Flat bones are, for example, skull bones, ribs, scapula, or pelvic bones. For example, WO 2015/144772 A1 teaches an eye socket covering grid for replacing a portion of an eye socket. Such implants must be mechanically stable, and thus these are frequently produced from a biocompatible metal, for example, titanium. Since the human anatomy is individually different, such implants are frequently patient-specifically produced, and/or are brought into the correct shape, for example, being bent by hand, during surgery. The use of metal implants adversely affects the selection of X-ray or MRI diagnostics of the implant site, however.

Patent application US 2019069984 A1 describes a polymer-based implant for replacing a bone, wherein a porous body is bonded to a non-porous material, so that the implant has a porous surface and a non-porous surface. The materials and layer thicknesses that are used are selected such that the implant can be brought into the desired shape during surgery, allowing the use of a metal support structure to be dispensed with. Due to such a polymer-based implant, X-rays or MRI can be used for the diagnostics of the implant site.

### BRIEF SUMMARY

Intraoperative adaptation of the implant geometry can limit the mechanical strength of an implant. Example aspects of the invention provide an implant which has a reduced or minimal adverse effect on the use of X-rays or MRI for the diagnostics of the implant site, and which is distinguished by high mechanical strength. Further example aspects of the invention provide a method for producing such an implant.

In an example embodiment, an implant is configured for replacing or filling a defect of a flat bone. The implant has a flat shape with a first side and an opposite second side. A “flat shape” is understood to mean both a planar surface and a curved surface. The implant has at least a first layer and a second layer. The first layer is formed entirely or predominantly of non-porous polyetheretherketone (PEEK) or polyethylene (PE), and has such a layer thickness that the shape of the implant is established by the shape of the first layer. An “established shape” is understood to mean that the implant cannot be plastically deformed without being damaged. The shape of the implant can therefore not be adapted during surgery by bending. The second layer is formed entirely or predominantly of a porous polymer which is not a polyaryletherketone or a polyetheretherketone. The second layer is arranged on the first side of the implant.

If the first layer is formed entirely or predominantly of non-porous polyethylene, preferably ultra high molecular weight polyethylene (UHMWPE) is selected as the material for this purpose.

The layered structure of the implant according to example aspects of the invention enables high mechanical strength, which is substantially provided by the non-porous layer. Due to the second layer, which is formed of a porous polymer, the ingrowth behavior of the implant into surrounding tissue is ensured. Due to the dimensionally stable first layer, there is no risk of the porous second layer becoming damaged due to mechanical overloading during the implantation process.

Preferably, the implant does not have a metal support structure. Due to the complete omission of such a metal support structure, diagnostics of the implant site by X-rays or MRI is completely unaffected.

Preferably, the second layer is formed entirely or predominantly of one of the following materials: polyethylene; polyphenylene sulfone; or polypropylene, particularly preferably of ultra-high molecular weight polyethylene (UHMWPE). These materials are distinguished by high biocompatibility and, in the porous form, offer good ingrowth behavior for surrounding tissue.

Preferably, the second layer is present in a pressed and fused granular form. As a result, a homogeneous porosity combined with high resistance to superficial damage of the second layer is achieved. Such a pressed and fused granular form can be obtained, for example, by using a mixture of starting particles, in which ten percent (10%) of the weight has a particle size less than one thousand micrometers (1000 μm), fifty percent (50%) of the weight has a particle size less than five hundred micrometers (500 μm), and ninety percent (90%) of the weight has a particle size less than four hundred and fifty micrometers (450 μm).

According to a preferred first example embodiment, a further layer is not arranged on the second side of the implant, so that the first layer formed of the non-porous polyetheretherketone (PEEK) is completely exposed. This example embodiment is particularly advantageous for the case in which tissue is not to grow onto the second side of the implant. This can be required, for example, in the case of a cranial implant. In such a cranial implant, the second side of the implant therefore forms the proximal side.

According to a preferred second example embodiment, the implant has a third layer on the second side, which is formed of the same material as the second layer arranged on the first side of the implant. In other words, the implant in this example embodiment has a middle first layer formed of non-porous polyetheretherketone (PEEK) or polyethylene (PE), on which a porous layer is arranged at least in some portions on each side. This example embodiment is advantageous for the case in which an ingrowth of tissue is desired on both sides of the implant, for example, in the case of an orbital floor implant.

Preferably, a porosity and/or surface roughness of the third layer differs from a porosity and/or surface roughness of the second layer. This enables the ingrowth behavior of tissue into the second and third layers to be intentionally influenced. A microporous structure thus improves the ingrowth behavior of connective tissue, while a macroporous structure promotes the ingrowth of hard tissue such as bone cells. The different porosity can be achieved, for example, due to the selection of the starting particle size. For example, a starting particle size of maximum one thousand micrometers (1000 μm) can be used to produce the second layer and a starting particle size of maximum seven hundred micrometers (700 μm) can be used to produce the third layer. Alternatively, the second and the third layers can have the same porosity and/or surface roughness. If the implant is in the form of an orbital floor implant, a more coarsely grained granular material can be used, for example, for the third layer, which forms the underside of the implant, than for the second layer, which forms the top side of the implant facing the eyeball.

Preferably, at least one portion of the first layer is not covered with a further layer. In other words, the non-porous polyetheretherketone (PEEK) or polyethylene (PE) is partially exposed, thus intentionally preventing the ingrowth of tissue in this exposed region.

The first layer formed of non-porous polyetheretherketone (PEEK) or polyethylene (PE) is preferably formed either over the entire surface or by multiple strips which are interconnected but separated from one another in some portions.

Thus, the first layer can have, for example, a peripheral edge which surrounds multiple strips which are separated from one another, wherein the strips and the peripheral edge are formed in one material or piece. In such an example embodiment, the second layer formed of the porous polymer can also extend across the intermediate spaces between the strips, so that some portions of the second layer are exposed on both sides. Due to such an example embodiment, the implant can be designed such that soft tissue can grow onto the implant on both sides of the implant, at least at defined points, without the need to apply a porous layer onto both sides of the first layer. As a result, the amount of foreign material implanted into the body can be kept low.

Preferably, the first layer formed of non-porous polyetheretherketone has at least one passage opening. As a result, an exchange of fluid and pressure compensation between the first side and the second side are made possible in the implanted state.

Preferably, the implant has at least one recess for accommodating a fixing element. The recess is formed in the first layer and/or in the second layer. The fixing element can be formed, for example, by a metal plate, which is preferably produced from titanium or stainless steel. In such an example embodiment, the implant can be fixed to the surrounding anatomy by screws. Alternatively or additionally, the fixing element can be designed as a suture anchor, so that the implant can be sutured to the surrounding anatomy.

Preferably, at least one of the layers is enriched with elements of at least one of the following materials: silver; strontium; magnesium; tricalcium phosphate; hydroxyapatite; molybdenum; calcium carbonate. Due to the antibacterial and bioactive effect of these substances, a likelihood of inflammation after the implantation process is reduced, and the ingrowth behavior into surrounding tissue is improved.

According to a preferred example embodiment, the implant is a cranial implant. In such an example embodiment, it is advantageous when the second side of the implant is the proximal side, i.e., the side facing the dura mater in the implanted state. In such an example embodiment it is also advantageous when the first side is covered with the layer formed of the porous polymer over the entire surface except for recesses—if present—for accommodating fixing elements. In such an example embodiment, the second side facing the dura mater is preferably not covered by a further layer, such that the non-porous polyetheretherketone is exposed on the second side of the implant.

According to a further preferred example embodiment, the implant is an eye socket implant, which is also referred to as an orbital implant. In such an example embodiment, it is advantageous when the second side of the implant is the side that faces the eyeball in the implanted state. On the portion on which the eyeball is to glide in the implanted state, either the non-porous polyetheretherketone or polyethylene is exposed, or a third microporous layer is provided. On the first side of the implant, the second layer formed of the porous polymer is applied at least in some portions, so that tissue can grow into the implant on this side.

Example aspects of the invention also provide a method for producing an implant which, in the produced state, has at least the features described above. The method is characterized by the following: providing the fully formed first layer formed of the non-porous polyetheretherketone (PEEK) or polyethylene (PE); activating at least a portion of the surface of the first layer with low-pressure plasma; and subsequently applying the second layer onto the correspondingly activated surface of the first layer by pressing on and fusing a granular material.

Activating the surface of the first layer considerably improves the adhesion of the second layer on the first layer. The specific configuration of the low-pressure plasma depends on the geometry of the first layer, wherein an applied maximum power of one hundred to four hundred (100-400) watts has proven effective in tests. Preferably, the process of pressing on the second layer is carried out immediately after the surface activation of the first layer, preferably by a die and a punch, the shape of which defines the implant geometry. In the process of pressing on, the pressure and temperature curve over time are preferably defined, so that a reproducible process is formed. The individual values for pressure and temperature over time depend on the implant geometry.

### BRIEF DESCRIPTION OF THE DRAWINGS

Exemplary embodiments of the invention are described in detail on the basis of the figures. Wherein:

FIG. 1 and FIG. 2 each show a view of an implant according to a first exemplary embodiment;

FIG. 3 and FIG. 4 each show a detailed view of the implant according to the first exemplary embodiment;

FIG. 5 shows a schematic side view of an implant according to a second exemplary embodiment;

FIG. 6 shows a schematic isometric view of an implant according to a third exemplary embodiment;

FIG. 7 shows a schematic top view of an implant according to a fourth exemplary embodiment;

FIG. 8 shows a schematic top view of an implant according to a fifth exemplary embodiment; and

FIG. 9 shows a schematic top view of an implant according to a sixth exemplary embodiment.

### DETAILED DESCRIPTION

Reference will now be made to embodiments of the invention, one or more examples of which are shown in the drawings. Each embodiment is provided by way of explanation of the invention, and not as a limitation of the invention. For example, features illustrated or described as part of one embodiment can be combined with another embodiment to yield still another embodiment. It is intended that the present invention include these and other modifications and variations to the embodiments described herein.

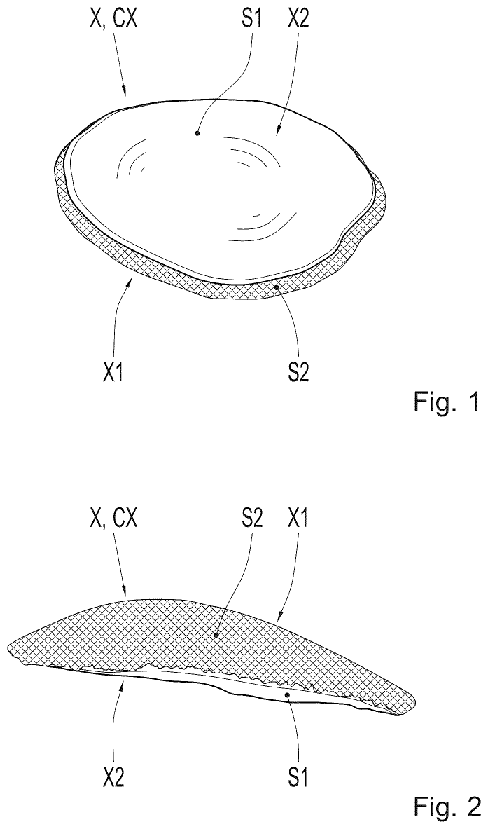

FIG. 1 and FIG. 2 each show a view of an implant X according to a first exemplary embodiment. The implant X is suitable for replacing or filling a defect of a flat bone. The first exemplary embodiment of the implant X shown in FIG. 1 and FIG. 2 is intended for use in the cranium, for example, as a cranial implant CX. The implant X has a flat curved shape with a first side X1 and an opposite second side X2. The implant X has a first layer S1 formed of a non-porous polyetheretherketone or non-porous polyethylene, preferably formed of ultra high molecular weight polyethylene. On the first side X1, the implant X has a second layer S2 formed of a porous polymer, which is attached on the first layer S1. The second layer S2 is formed entirely or predominantly of polyethylene, polyphenylene sulfone or polypropylene, particularly preferably of ultra-high molecular weight polyethylene (UHMWPE). The first layer S1 is so thick that it is not possible to shape the implant X during surgery. In other words, the first non-porous layer S1 is so thick that the first layer S1 is dimensionally stable, such that the first layer S1 cannot be plastically deformed to adapt the implant geometry without damaging the second porous layer S2. The minimum wall thickness of the first layer S1 necessary for this mechanical behavior depends on the size and shape of the implant X. In the example shown in FIG. 1, the implant X is approximately ten centimeters (10 cm) long and ten centimeters (10 cm) wide, and has a curved surface shape. If the first layer S1 is produced from polyetheretherketone or ultra-high molecular weight polyethylene, the wall thickness of the first layer S1 is two millimeters (2 mm) to four millimeters (4 mm) on average given this geometry. Depending on the mechanical load on the implant X, the first layer S1 can also have a greater mean wall thickness, for example, six millimeters (6 mm).

The second layer S2 is present in a pressed and fused granular form. As a result, a homogeneous porosity of the second layer S2 combined with high resistance to superficial damage is obtained. Due to the homogeneous porosity, an ingrowth of tissue into the second layer S2 is made possible, whereas, due to the non-porous design of the first layer S1, an ingrowth of tissue into the first layer S1 is prevented.

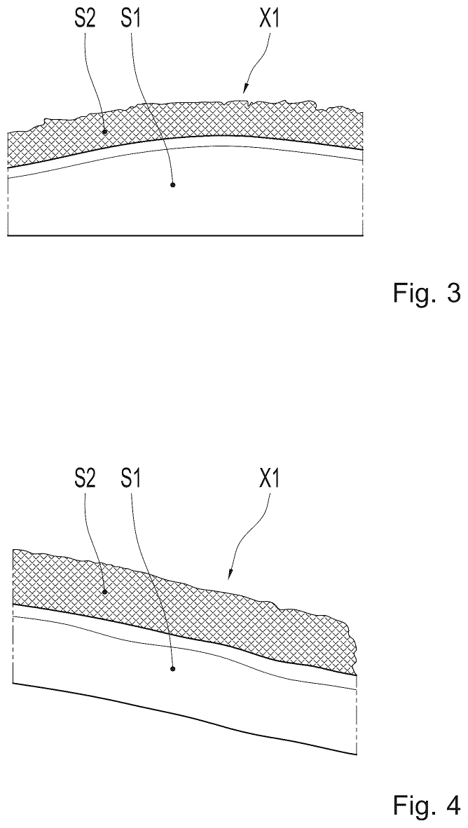

FIG. 3 and FIG. 4 each show a detailed view of the implant X according to the first exemplary embodiment. In order to produce such an implant X, initially the first layer S1 is produced, for example, by a forming press or by a generative/additive manufacturing process, also known as 3D printing. In particular by using a generative manufacturing process, the first layer S1 can be produced precisely according to the individual needs of a patient. Then the first layer S1 is reworked if necessary, in order to remove burrs or supporting structures from the preceding production step. At least a portion of the first layer S1 is subsequently activated by using a low pressure plasma to generate a particularly adhesive surface. Immediately thereafter, the second layer S2 is applied onto the previously activated surface of the first layer S1, specifically by pressing on and fusing a granular material. In this way, the second layer S2 can be securely applied onto the first layer S1 without the need for an additional adhesive or the like.

It becomes clear from the illustration according to FIG. 3 that the second layer S2 can be considerably thinner than the first layer S1. FIG. 4 shows an example embodiment in which the second layer S2 is as thick as the first layer S1.The specific thickness of the porous second layer S2 can be selected according to the intended use and the specific needs of the patient. The second layer S2 can have an inhomogeneous layer thickness, such that a thicker second layer S2 is present at one point of the implant X than at another point. A thickness of the second layer S2 between one millimeter (1 mm) and six millimeters (6 mm) has proven advantageous in tests. A typical overall thickness of the implant X having a two-layer structure with a first layer S1 and a second layer S2 is four millimeters (4 mm) to eight millimeters (8 mm).

The representation of the porous second layer S2 in the figures is abstracted, since a representation of the actual porosity in patent drawings is not reliably recognizable and reproducible.

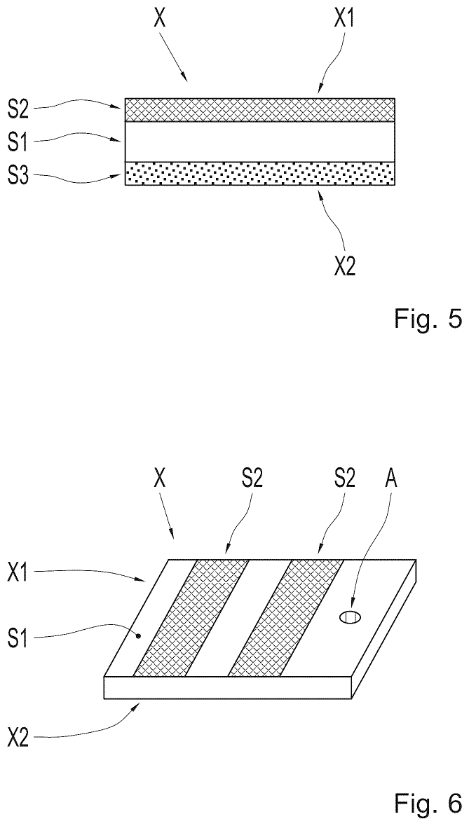

FIG. 5 shows a schematic side view of an implant X according to a second exemplary embodiment. In this example embodiment, the implant X has a porous layer both on the first side X1 and on the opposite second side X2. To this end, the second layer S2 is arranged on the first side X1, whereas a third layer S3 is arranged on the second side X2. The third layer S3 is made of the same material as the second layer S2. However, the third layer S3 has a different porosity than the second layer S2, as is indicated in the illustration according to FIG. 5. The different porosity is achieved by using a different granule size. The first layer S1 lies between the second layer S2 and the third layer S3, so that the implant X has a three-layer sandwich structure.

FIG. 6 shows a schematic isometric view of an implant X according to a third exemplary embodiment. In this example embodiment, multiple portions of the first layer S1 are not covered by a further layer, such that portions of the first layer S1 are exposed on both sides X1, X2 of the implant X. On the first side X1, only two strips of the second layer S2 are applied on the first layer S1. The second layer S2, which is arranged in a strip-shaped manner, has a thin wall, for example, with a layer thickness of only one millimeter (1 mm). A through-opening A is provided in a portion of the first layer S1 that is not covered on either side. Due to the through-opening, an exchange of fluid and pressure compensation between the first side X1 and the second side X2 are made possible in the implanted state. The implant X can have multiple such through-openings A.

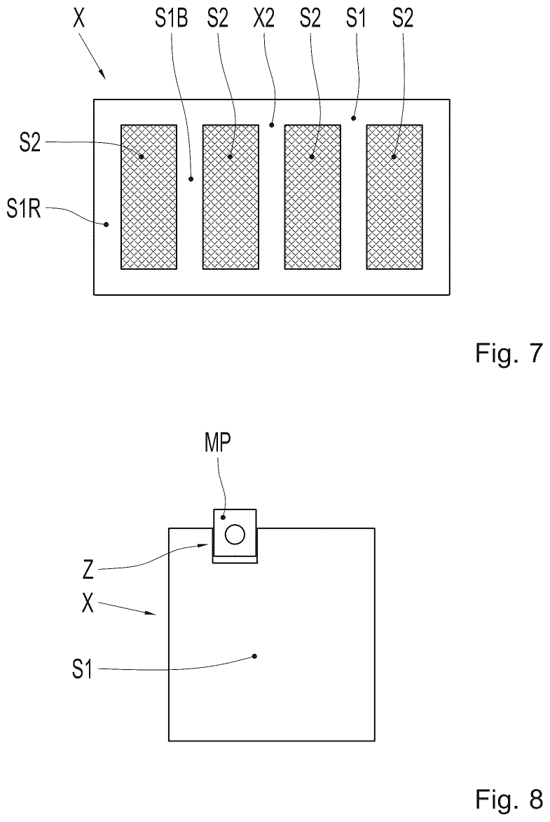

FIG. 7 shows a schematic top view of the second side X2 of an implant X according to a fourth exemplary embodiment. In this example embodiment, the first layer S1 has a peripheral edge S1R and multiple interconnected strips S1B. The first layer S1 has multiple voids between the strips S1B. In contrast to the exemplary embodiments shown in FIG. 1 through FIG. 6, the first layer S1 is therefore not formed over the entire surface. The second layer S2 is arranged on the first side X1 (not visible in FIG. 7) of the implant X and is also visible from the second side X2 through the voids in the first layer S1. In this example embodiment, the second layer S2 is therefore accessible from both sides in some portions. In such an example embodiment, a third layer S3 formed of a porous polymer can be arranged on at least some portions of the second side X2, as is schematically shown in FIG. 5. For the sake of clarity, such an example variant is not explicitly shown in the figures.

FIG. 8 shows a schematic top view of an implant X according to a fifth exemplary embodiment. A recess Z, which is designed to accommodate a fixing element MP, is provided on the edge of the implant X. The fixing element MP can be, for example, a metal plate that has a hole for accommodating a fastening screw or a strand of suture. The implant X can have multiple such fixing element MP, which are arranged in multiple recesses Z distributed on the edge of the implant X.

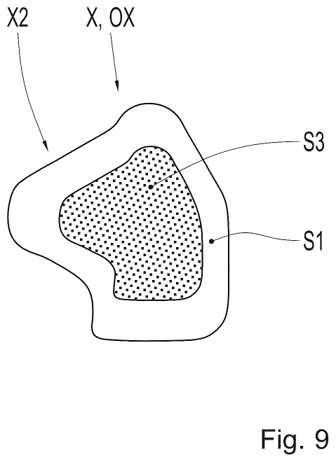

FIG. 9 shows a schematic top view of the second side X2 of an implant X according to a sixth exemplary embodiment. The implant X is in the form of an eye socket implant OX. A third layer S3 formed of a porous polymer is arranged on a portion of the second side X2. The non-porous first layer S1 is exposed on the edge region of the implant X, i.e., is not covered by a porous layer. The second layer S2 is formed on at least some portions of the first side X1 (not visible in FIG. 9).

Modifications and variations can be made to the embodiments illustrated or described herein without departing from the scope and spirit of the invention as set forth in the appended claims. In the claims, reference characters corresponding to elements recited in the detailed description and the drawings may be recited. Such reference characters are enclosed within parentheses and are provided as an aid for reference to example embodiments described in the detailed description and the drawings. Such reference characters are provided for convenience only and have no effect on the scope of the claims. In particular, such reference characters are not intended to limit the claims to the particular example embodiments described in the detailed description and the drawings.

### LIST OF REFERENCE SIGNS

X implant
CX cranial implant
OX eye socket implant
X1 first side
X2 second side
S1 first layer
S1B strip
S1R edge
S2 second layer
S3 third layer
A through-opening
Z recess
MP fixing element

1-16. (canceled)

17. An implant (X) for replacing or filling a defect of a flat bone, the implant (X) having a flat shape with a first side (X1) and an opposite second side (X2), the implant (X) comprising:
a first layer (S1) formed of non-porous polyetheretherketone or polyethylene, the first layer (S1) having a layer thickness such that a shape of the implant (X) is established by a shape of the first layer (S1); and
in at least some portions on the first side (X1), a second layer (S2) formed of a porous polymer that is not polyaryletherketone or polyetheretherketon.

18. The implant (X) of claim 17, wherein the implant (X) does not comprise a metal support structure.

19. The implant (X) of claim 17, wherein the second layer (S2) is formed entirely or predominantly of polyethylene, polyphenylene sulfone, or polypropylene.

20. The implant (X) of claim 19, wherein one or both of the first layer (S1) and the second layer (S2) is formed of ultra-high molecular weight polyethylene.

21. The implant (X) of claim 17, wherein the second layer (S2) is a pressed and fused granular layer.

22. The implant (X) of claim 17, wherein a further layer is not arranged on the second side (X2) of the implant (X) such that the first layer (S1) is exposed.

23. The implant (X) of claim 17, further comprising a third layer (S3) on the second side (X2) of the implant (X), the third layer (S3) formed of the same material as the second layer (S2).

24. The implant (X) of claim 23, wherein a porosity and/or a surface roughness of the third layer (S3) differs from a porosity and/or a surface roughness of the second layer (S2).

25. The implant (X) of claim 17, wherein at least a portion of the first layer (S1) is not covered by a further layer (S2, S3).

26. The implant (X) of claim 17, wherein the first layer (S1) is disposed over an entire surface of the second side (X2) or is formed by interconnected strips (S1B).

27. The implant (X) of claim 17, wherein the first layer (S1) defines at least one through-opening (A).

28. The implant (X) of claim 17, wherein at least one recess (Z) for accommodating a fixing element (MP) is formed in the first layer (S1) and/or in the second layer (S2).

29. The implant (X) of claim 17, wherein at least one of the layers (S1, S2, S3) of the implant (X) is enriched with: silver; strontium; magnesium; tricalcium phosphate; hydroxyapatite; molybdenum; calcium carbonate; or combinations thereof.

30. The implant (X) of claim 17, wherein the implant (X) is a cranial implant (CX).

31. The implant (X) of claim 17, wherein the implant (X) is an eye socket implant (OX).

32. A method for producing the implant (X, CX, OX) of claim 17, comprising:
providing a fully formed first layer (S1);
activating at least a portion of a surface of the first layer (S1) with low pressure plasma; and
applying the second layer S2 onto the activated surface of the first layer (S1) by pressing on and fusing a granular material.
